(19) United States
(12) Patent Application Publication
(10) Pub. No.: US 2007/0073205 A1
(43) Pub. Date: Mar. 29, 2007

(54) Digit Splint

(75) Inventors: James E. Hull, Knoxville, TN (US); Randel B. Holmes, Knoxville, TN (US); Steve Bryant, Knoxville, TN (US); Tracy Hull, Knoxville, TN (US)

Correspondence Address: PITTS AND BRITTIAN P C, P O BOX 51295, KNOXVILLE, TN, 37950-1295, US

(73) Assignee: Prognotions, LLC, Knoxville, TN (US)

(21) Appl. No.: 11/458,830

(22) Filed: Jul. 20, 2006

Related U.S. Application Data
(60) Provisional application No. 60/720,660, filed on Sep. 26, 2005.

(51) Int. Cl. A61F 5/00

(52) U.S. Cl. 602/22; 602/5

(57) ABSTRACT

A digit splint, for partially immobilizing a digit, such as a finger or a toe, for therapeutic purposes. The digit splint of the present invention is of laminate construction having a base layer, a semi-rigid or rigid layer, and a covering layer. The base layer carries an adhesive for securing the splint to the digit. The semi-rigid or rigid layer, in one embodiment, is constructed of a heavier gauge polyester, or plastic, film. The covering layer is adhered to the base layer, with the semi-rigid or rigid layer sandwiched there between, and provides a, preferably, water-proof covering for the digit splint.

### CROSS-REFERENCE TO RELATED APPLICATIONS

This application claims of the benefit of the filing date of provisional patent application Ser. No. 60/720,660 filed on Sep. 26, 2005.

### STATEMENT REGARDING FEDERALLY SPONSORED RESEARCH OR DEVELOPMENT

Not Applicable

### BACKGROUND OF THE INVENTION

1. Field of Invention

This invention pertains to devices for immobilizing human appendages. More particularly, this invention pertains to a splint for partially immobilizing a digit, particularly a finger or toe, for therapeutic purposes.

2. Description of the Related Art

When a finger, or other digit, such as a toe, requires immobilization for medical purposes, it is common to use a metal splint or brace. It will be appreciated by those skilled in the art that the state of the art metal brace is comprised of a generally flat metal plate that has been bent into a U shape so as to cover the tip of the finger and extend for a partial length of the finger. This brace is then taped into place. The result is a bulky brace that is unattractive and is unwieldy, and potentially dangerous, for athletes participating in certain types of sports. Another problem is that the distal end of the finger is held rigidly immobilized. It is believed that it is desirous, from a recuperative standpoint, for the braced finger to be provided with some, albeit limited, flexibility. This limited flexibility would also be advantageous to, and increase the dexterity of, athletes participating in a sporting event.

What is missing from the art is a small, lightweight, and inexpensive finger, or other digit, brace that wraps around the distal end of the digit to be braced, so as to partially immobilize the finger in a manner that allows the wearer to participate in sporting activities. What is additionally missing from the art is a small, lightweight, and inexpensive finger brace that preserves some limited flexibility of the finger being braced.

### BRIEF SUMMARY OF THE INVENTION

According to one embodiment of the present invention, a digit splint, for partially immobilizing a digit, such as a finger or a toe, for therapeutic purposes, is of laminate construction having a base layer, a semi-rigid member, and a covering layer. The base layer carries an adhesive for securing the splint to the digit. The semi-rigid or rigid member, in one embodiment, is constructed of a heavier gauge material. The semi-rigid or rigid member is elongated and is narrower than either the base layer or the covering layer. In the preferred embodiment, the semi-rigid or rigid member includes a region, disposed approximately equidistant from the distal ends of the semi-rigid or rigid member that is narrower than the remaining portions of the semi-rigid or rigid member. In the preferred embodiment, the distal ends of the semi-rigid or rigid member are of substantially uniform width. The covering layer is adhered to the base layer, with the semi-rigid or rigid member sandwiched there between, and provides a, preferably, non-woven water-proof covering for the digit splint. The covering layer constitutes the outer layer when the digit splint is worn on a digit such as a finger.

The digit splint is preferably elongated so as to wrap around the digit. For purposes of discussion and illustration, a finger will be presented as an exemplary digit. However, it should be understood that the digit splint could also be dimensioned so as to be utilized on a toe. In this regard, the digit splint comprises a first region for covering the palmar portion of the digit and a second region for covering the dorsal portion of the digit. The first region and the second region are interconnected by means of a narrow band that extends over the tip of the digit. It will be understood that the first region, second region and the narrow band are, preferably, integral. In the preferred embodiment the distal ends of each of the first region and the second region are wider than the proximal ends of the first and second regions and are dimensioned so as to substantially overlap when the digit splint is applied to the finger.

### BRIEF DESCRIPTION OF THE SEVERAL VIEWS OF THE DRAWINGS

The above-mentioned features of the invention will become more clearly understood from the following detailed description of the invention read together with the drawings in which:

FIG. 1 is a plan view of a lightweight digit splint constructed in accordance with the present invention.

FIG. 2 is a plan view of the internal semi-rigid or rigid member used for providing stability in the lightweight digit splint of the present invention.

FIG. 3 is an exploded view of the digit splint of the present invention.

FIG. 4 through FIG. 8 represent perspective views illustrating the steps of applying the digit splint of the present invention to a finger.

FIG. 9 and FIG. 10 represent perspective views illustrating the application of the digit splint of the present invention to a finger such that the semi-rigid or rigid member extends across the distal end of the finger and forms a “u” shape on each side of the finger to provide more rigid support for an injured finger.

### DETAILED DESCRIPTION OF THE INVENTION

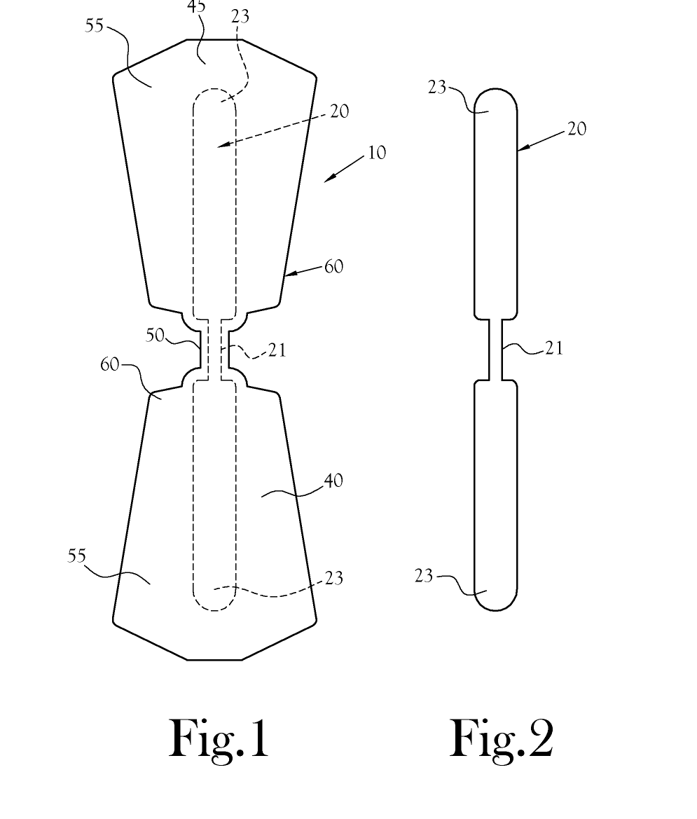

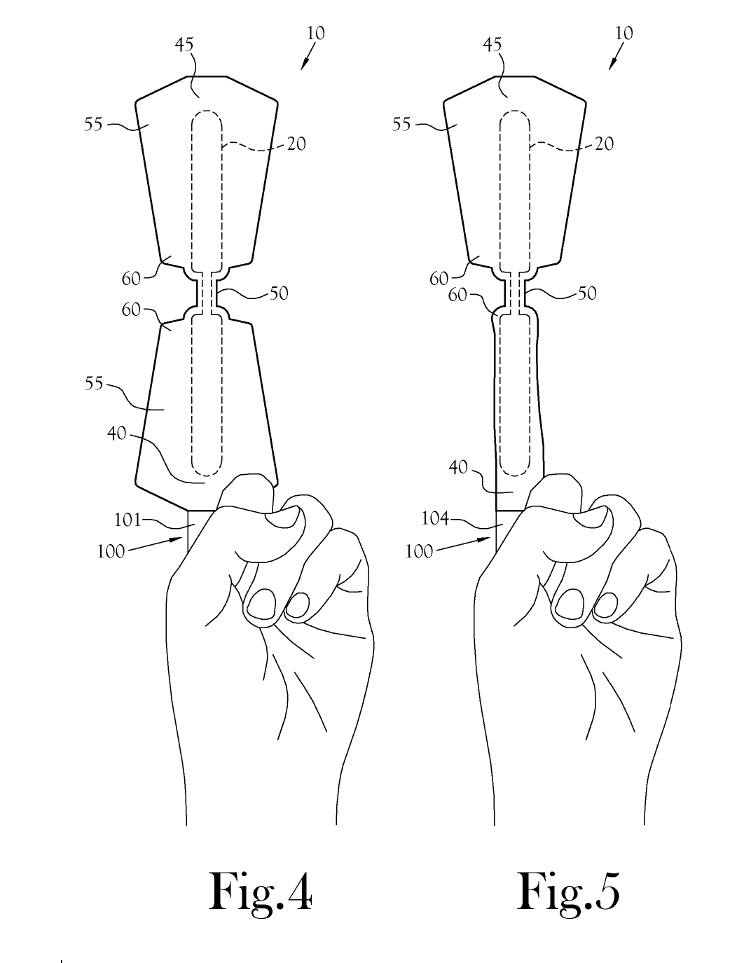

A digit splint constructed in accordance with the present invention is designated generally as 10 in the Figures. According to one embodiment of the present invention, digit splint 10 is designed for partially immobilizing a digit, such as finger 100 seen in FIGS. 4 through 8, or a toe (not illustrated), for therapeutic purposes. Referring to FIGS. 1 through 3, digit splint 10 is of laminate construction having a base layer 15, a semi-rigid or rigid member 20, and a covering layer 25. The base layer 15 is preferably constructed of an adhesive based material that will readily and releasably adhere to skin in a non-damaging or destructive manner. In other words, while the material should be selected such that it will securely adhere to the skin of the finger 100, it should be removable from the skin without damaging the skin. While base member 15 could be constructed of a woven medical tape, it is preferably constructed of a non-woven tape such as, though not limited to, Sontara®, also marketed as DuPont™ Softesse™. It will be appreciated that the medical tape that comprises the base layer 15 carries a first adhesive 30 for securing digit splint 10 to finger 100. The base layer 15 also carries, on an oppositely disposed side from the first adhesive 30, a second adhesive 35. This second adhesive 35 serves to adhere the remaining components of digit splint 10 to the base layer 15.

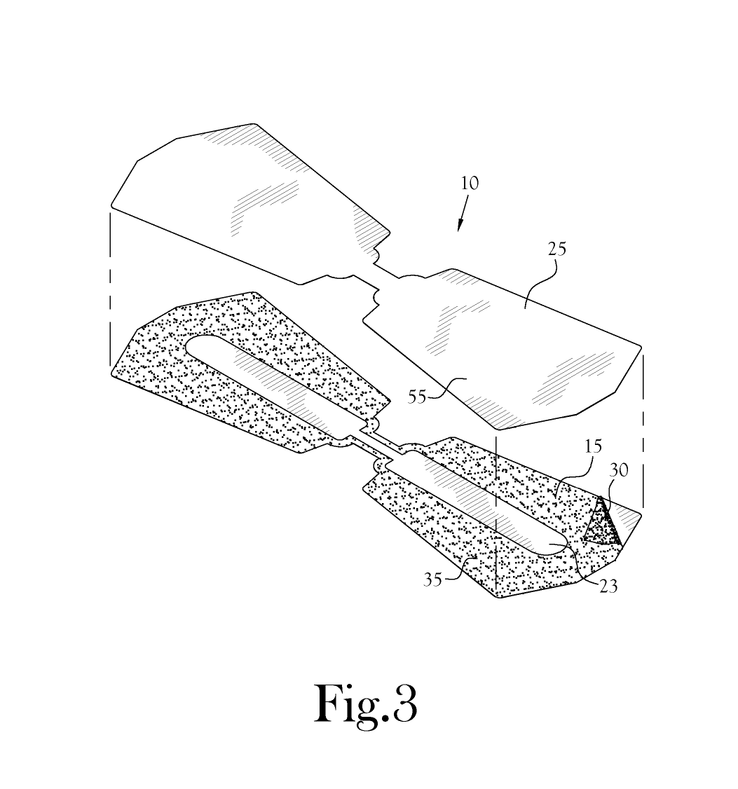

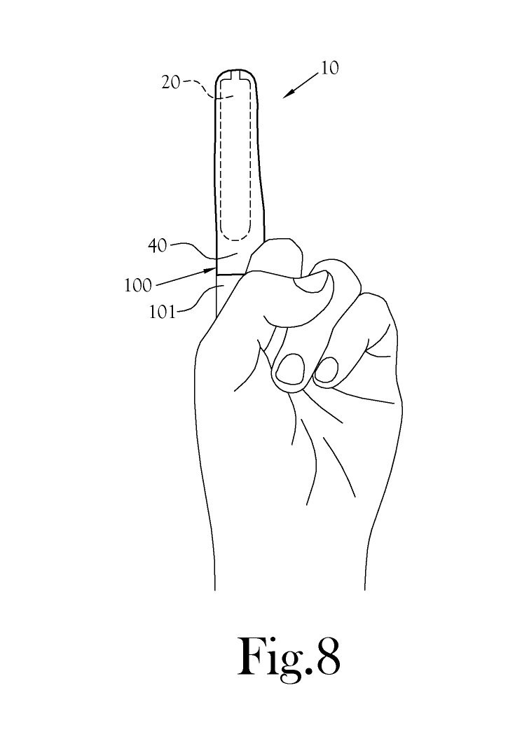

A semi-rigid or rigid member 20, illustrated in FIG. 2, is adhesively carried by base layer 15 for providing stiffness to the digit splint 10 thereby partially immobilizing the digit when the digit splint 10 is applied to the finger, as seen in FIG. 8. Adhesive can be used to secure member 20 to layer 15 as necessary or desired. In this regard, the semi-rigid or rigid member 20 is, preferably, constructed of a heavy gauge material. As illustrated best in FIG. 2, the semi-rigid or rigid member 20 is elongated and, as illustrated in FIGS. 1 and 3, is narrower than either the base layer 15 or the covering layer 25. In the preferred embodiment, the semi-rigid or rigid member 20 includes a narrow mid-region 21, disposed approximately equidistant from the distal ends 23 of the semi-rigid or rigid member 20 that is narrower than the remaining portions of the semi-rigid or rigid member 20. In the preferred embodiment, the distal ends 23 of the semi-rigid or rigid member are of substantially uniform width. It will be appreciated that when applied to the finger 100, one distal end 23 of semi-rigid or rigid member 20 will be positioned proximate a palmar side of finger 100 while the opposite distal end 23 of the semi-rigid or rigid member will be positioned proximate a dorsal side of finger 100 with the narrow mid-region 21 being proximate, and overlapping, the tip of finger 100.

The covering layer 25 is adhered to the base layer 15 by means of second adhesive 35, with the semi-rigid or rigid member 20 sandwiched there between. It will be appreciated that the covering layer 25 is dimensionally similar to base layer 15 and has a substantially similar geometric configuration. In one embodiment, the covering layer 25 is a thin gauge polyethylene film for creating the outer layer of the digit splint 10 and is preferably moisture resistant, or even water-proof. It will be appreciated that other materials, including non-woven fabric and other materials, exist which are suitable for use to accomplish the covering layer 25 of the present invention. The covering layer 25 can be printed in a variety of colors and with a variety of indicia. In this regard, it should be appreciated that the covering layer 25 constitutes the outer layer of the digit splint 10 when the digit splint 10 is worn on a digit such as finger 100.

As discussed above, it should be appreciated that while the digit splint 10 is described and illustrated as being applied to a finger, the digit splint 10 could also be dimensioned so as to be utilized on a toe. The digit splint 10 is preferably elongated, having a length that exceeds its width, so as to cover the palmar region of the digit as well as the dorsal region of the digit, extending over the tip of the digit, such as finger 100. In this regard, the digit splint 10 comprises a first region 40 for covering the palmar portion 101 of finger 100 and a second region 45 for covering the dorsal portion 104 of finger 100. The first region 40 and the second region 45 are interconnected by means of a narrow band 50 that extends over the tip of finger 100. It will be understood that the first region 40, second region 45 and the narrow band 50 are, preferably, integral. In the preferred embodiment the distal ends 55 of each of the first region 40 and the second region 45 are wider than the proximal ends 60 of the first region 40 and second region 45 and are dimensioned so as to substantially overlap when the digit splint is applied to the finger. The overlapping action of the distal ends 55 serves to stabilize and reinforce the action of the digit splint 10 in partially immobilizing the digit. It being understood that the term “partially immobilizing” refers to a partial restriction of the flexing of the digit at the joint between the proximal phalanx and the middle phalanx, and at the joint between the middle phalanx and the distal phalanx.

Application of the digit splint 10 to finger 100 is illustrated in step-wise fashion in FIGS. 4-8. In FIG. 4, the first region 40 has been adhesively secured to the palmar region 101 of finger 100 such that a portion of the length of the semi-rigid or rigid member 20 extends along the palmar length of finger 100. The digit splint 10 is positioned such that the narrow band 50 along with the narrow mid region 21 are each proximate the tip of the finger 100. It will be appreciated that the digit splint can be provided with a removable covering (not illustrated) for covering the first adhesive 30 prior to application of the digit splint 10 to a digit. The side members of the first region 40 are then wrapped around the length of finger 100 and adhesively secured to finger 100 as seen in FIG. 5.

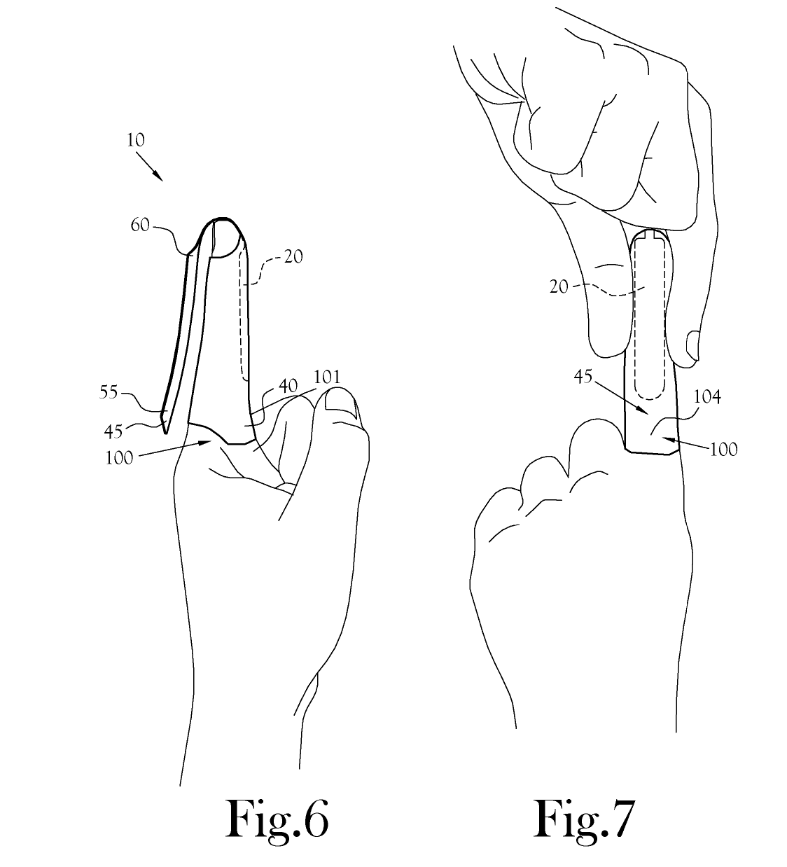

The second region 45 is then folded over so as to cover, and be adhesively secured to, the dorsal portion 104 of finger as best seen in FIG. 6. The side members of the second region 45 are then wrapped around the length of finger 100 so as to overlap the side portions of the first region 40. As seen in FIG. 8, when applied in this manner, the digit splint substantially conforms to the profile of the finger 100, thus substantially reducing, and effectively eliminating the bulk of state of the art splints.

It will be recognized by those skilled in the art that the above described components of the digit splint 10 can be combined in the process known as rotary die cutting. In this regard, as understood by those skilled in the art, raw materials, stored on spools is unwound through nip rollers and then die cut. In this regard, a first die cutter cuts semi-rigid or rigid member 20 prior to being island placed between the base layer 15 and the covering layer 20. All three materials are then fed through a final nipping process and then die-cut to the final desired shape of the digit splint 10.

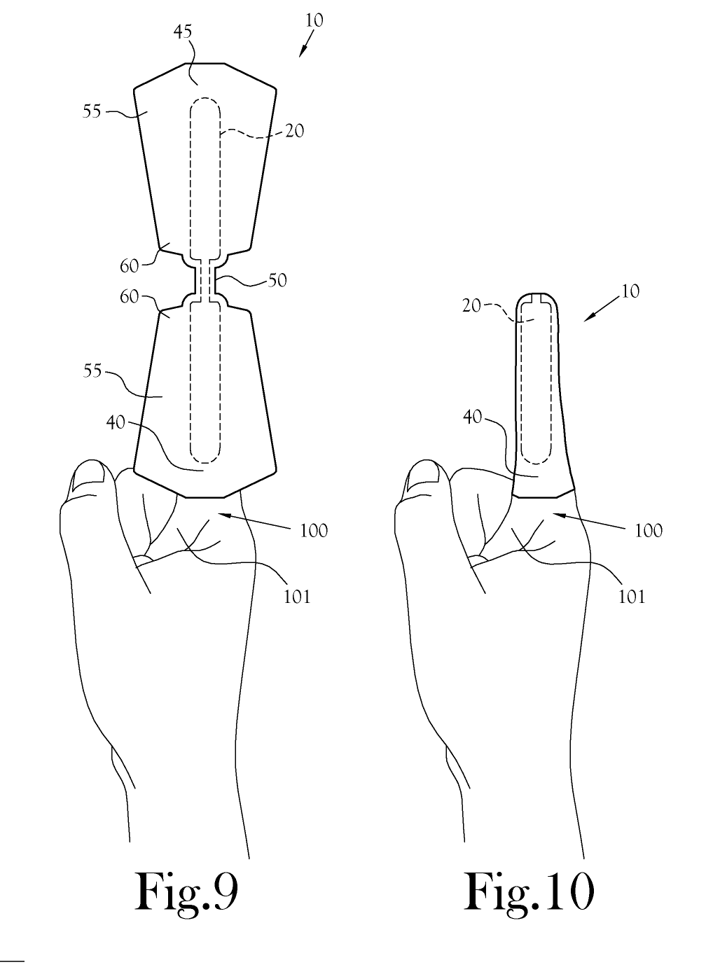

In another application of the invention the semi-rigid or rigid member can be applied to the sides of the finger to provide more rigid support as is shown in FIGS. 9 and 10. FIG. 9 illustrates the step prior to securing the splint to the digit with the splint rotated ninety degrees from the position shown in FIG. 4. The semi-rigid or rigid member 20 can be applied to the finger such that the narrow band 50 extends across the distal end of the finger and the semi-rigid or rigid member 20 forms a “u” shape on both side portions of the finger. By rotating the member 20 ninety degrees from the application shown in FIG. 7 to the position shown in FIG. 9, the splint can be used for collateral ligament sprains. Moreover, the application of the splint enhances the restriction of the finger motion and thus provides stronger support or more rigid support for an injured finger.

While the present invention has been illustrated by description of several embodiments and while the illustrative embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept

Having thus described the aforementioned invention, we claim:

1. A digit splint for partially restricting flexing of a digit, said digit splint comprising:
a laminar tape body having a first region for covering a palmar portion of the digit and a second region for covering a dorsal portion of the digit, said first region and said second region being connected by a narrow band member, wherein said first region, said second region and said narrow band member are of integral construction;
an elongated member disposed within said laminar tape body for providing stiffness to said laminar tape body; wherein said laminar tape body is adhesively secured to the digit by placing said first region proximate the palmar portion of the digit such that said narrow band member is proximate a tip of the digit, and folding said second region over the dorsal portion of the digit, said elongated member being at least semi-rigid.

2. The digit splint of claim 1 wherein each of said first region and said second regions include proximal ends proximate said narrow band member and further include distal ends in spaced relation from said narrow band member, wherein said distal ends are dimensioned such that said distal ends are wider than said proximal ends such that at least a portion of said distal end of said second region overlaps at least a portion of said distal end of said first region when said digit splint is applied to the digit.

3. The digit splint of claim 1 wherein said laminar tape body comprises a base layer and a covering layer wherein said elongated member is placed between said base layer and said covering layer.

4. The digit splint of claim 1 wherein said base layer is formed from a non-woven tape having a first adhesive on a surface for adhesively securing said digit splint to the digit and a second adhesive on a second surface for securing said elongated member and said covering layer to said base member.

5. The digit splint of claim 1 wherein said elongated member is constructed from a heavy gauge polyester film.

6. The digit splint of claim 1 wherein said elongated member includes first and second distal ends and narrow mid-region disposed approximately equidistant from said first and second distal ends.

7. The digit splint of claim 1 where said covering layer is constructed from a thin gauge polyethylene film for creating a moisture resistant outer covering for said digit splint.

8. A digit splint for partially restricting flexing of a digit, said digit splint comprising:
a base layer having first and second regions, wherein each of said first and second regions have distal ends and proximal ends, said distal ends being wider than said proximal ends such that at least a portion of said distal end of said second region overlaps at least a portion of said distal end of said first region when said digit splint is applied to the digit;
a narrow band member for interconnecting said proximal ends, said first region, said second region and said narrow band member being integral with one another;
an elongated member adhesively secured to said base layer for providing stiffness to said digit splint, said elongated member being at least semi-rigid;
a covering layer adhesively secured to said base layer such that said covering layer covers said elongated member, wherein said covering layer has a perimeter configuration substantially identical to said base layer;
wherein said digit splint is adhesively secured to the digit by placing said first region proximate a palmar portion of the digit such that said narrow band member is proximate a tip of the digit, and folding said second region over a dorsal portion of the digit.

9. The digit splint of claim 8 wherein said base layer is formed from a non-woven tape having a first adhesive on a surface for adhesively securing said digit splint to the digit and a second adhesive on a second surface for securing said elongated member and said covering layer to said base member.

10. The digit splint of claim 8 wherein said elongated member is constructed from a heavy gauge polyester film.

11. The digit splint of claim 8 wherein said elongated member includes first and second distal ends and narrow mid-region disposed approximately equidistant from said first and second distal ends.

12. The digit splint of claim 8 where said covering layer is constructed from a thin gauge polyethylene film for creating a moisture resistant outer covering for said digit splint.

13. The digit splint of claim 8 where said covering layer is constructed from a non-woven material for creating a moisture resistant outer covering for said digit splint.

14. A digit splint for partially restricting flexing of a digit, said digit splint comprising:
a laminar tape body having a first region for covering a side portion of the digit and a second region for covering the opposite side portion of the digit, said first region and said second region being connected by a narrow band member, wherein said first region, said second region and said narrow band member are of integral construction;
an elongated member disposed within said laminar tape body for providing stiffness to said laminar tape body; wherein said laminar tape body is adhesively secured to the digit by placing said first region proximate a first side portion of the digit such that said narrow band member is proximate a tip of the digit, and folding said second region over the opposite side portion of the digit, said elongated member being at least semi-rigid.
